(19) United States
(12) Patent Application Publication
(10) Pub. No.: US 2003/0225530 A1
(43) Pub. Date: Dec. 4, 2003

(54) Forensic investigations

(75) Inventors: Alexandra Louise Lowe, Birmingham (GB); Andrew James Urquhart, Birmingham (GB); Ian Evett, Birmingham (GB); Andrew John Hopwood, Birmingham (GB)

Correspondence Address: MERCHANT & GOULD PC, P.O. BOX 2903, MINNEAPOLIS, MN, 55402-0903, US

(73) Assignee: The Secretary of State for the Home Department, Birmingham (GB)

(21) Appl. No.: 10/360,838

(22) Filed: Feb. 6, 2003

Related U.S. Application Data
(63) Continuation of application No. 09/624,432, filed on Jul. 24, 2000, now abandoned.

(30) Foreign Application Priority Data
Jul. 23, 1999 (GB) 9917309.8

(51) Int. Cl. C12Q 1/68; G06F 19/00; G01N 33/48; G01N 33/50

(52) U.S. Cl. 702/20; 435/6

(57) ABSTRACT

The invention aims to provide additional information of the identity or source of DNA sample, particularly an ethnic characteristic, such as the ethnic grouping, of the person who is the source of the DNA sample.

The invention provides a method of obtaining information about the nature of a physical characteristic of the source of a sample from a number of possibilities for that physical characteristic, the method comprising

analysing at least part of the DNA in the sample, the analysis determining the presence and/or identity of one or more variations at one or more locations of the DNA;

providing a database containing information on the presence and/or identity of the one or more variations at the one or more locations of the DNA for a plurality of reference samples, the nature of the physical characteristic being known for the reference samples;

for one or more of the possible natures of the physical characteristic, taking at least some of the reference samples having a common nature for the physical characteristic together to give a grouping and considering the frequency of occurrence of the combination of the presence and/or identity of the one or more variations at the one or more locations of the DNA for the sample in that grouping having a common nature of the physical characteristic;

the frequency of occurrence being used to predict information relating to the nature of the physical characteristic of the source of the sample.

This invention concerns improvements in and relating to forensic investigations, particularly, but not exclusively, to using DNA based investigations to predict a physical characteristic of a samples source, and more particularly, but not exclusively to techniques for investigating or predicting the ethnic background of a DNA source.

In a variety of situations it is desirable to be able to obtain as much information as possible about the identity or source of a DNA sample. Such situations include analysis of crime scene samples where it is helpful to obtain details of the potential source of that sample with a view to tracing the samples source and/or linking a sample from a possible source to the crime scene sample and/or discounting a link between a sample from a possible source and the crime scene sample.

Forensic science already uses a variety of such techniques, such as single nucleotide polymorphisms, to compare the DNA characteristic of a sample with a sample from a known person. These techniques concern variations in the DNA on an individual basis, however. Additionally they do not allow any prediction to be made about the source of a DNA sample, for instance a crime scene sample, for instance a physical characteristic of the individual who generated the DNA sample.

According to a first aspect of the invention we provide a method of obtaining information about the nature of a physical characteristic of the source of a sample from a number of possibilities for that physical characteristic, the method comprising

analysing at least part of the DNA in the sample, the analysis determining the presence and/or identity of one or more variations at one or more locations of the DNA;

providing a database containing information on the presence and/or identity of the one or more variations at the one or more locations of the DNA for a plurality of reference samples, the nature of the physical characteristic being known for the reference samples;

for one or more of the possible natures of the physical characteristic, taking at least some of the reference samples having a common nature for the physical characteristic together to give a grouping and considering the frequency of occurrence of the combination of the presence and/or identity of the one or more variations at the one or more locations of the DNA for the sample in that grouping having a common nature of the physical characteristic to obtain the information about the nature of the physical characteristic of the source of the sample.

The first aspect may further provide that the frequency of occurrence is used to predict information relating to the nature of the physical characteristic of the source of the sample.

According to a second aspect of the invention we provide a method of obtaining information about the nature of a physical characteristic of the source of a sample from a number of possibilities for that physical characteristic, the method comprising

analysing at least part of the DNA in the sample, the analysis determining the presence and/or identity of one or more variations at one or more locations of the DNA;

providing a database containing information on the presence and/or identity of the one or more variations at the one or more locations of the DNA for a plurality of reference samples, the nature of the physical characteristic being known for the reference samples;

for one or more of the possible natures of the physical characteristic, taking at least some of the reference samples having a common nature for the physical characteristic together to give a grouping and considering the frequency of occurrence of the combination of the presence and/or identity of the one or more variations at the one or more locations of the DNA for the sample in that grouping having a common nature of the physical characteristic;

the frequency of occurrence being used to predict information relating to the nature of the physical characteristic of the source of the sample.

The physical characteristic may be the ethnic characteristic of the sample's source, particularly the ethnic character of the person who is the sample's source.

Preferably it is the identity of the potential variation which is considered.

Preferably it is the frequency of occurrence of those variations with ethnic characteristics which is considered.

Preferably the nature of the physical characteristic, for instance ethnic characteristic, is recorded in the database.

According to a third aspect of the invention we provide a method of obtaining information about the ethnic characteristic of a person who is the source of a sample, from a number of possible ethnic characteristics, the method comprising

analysing at least part of the DNA in the sample, the analysis determining the identity of one or more variations at one or more locations of the DNA;

providing a database containing information on the identity of the one or more variations at the one or more locations of the DNA for a plurality of reference samples taken from people whose ethnic characteristic is known and recorded in the database;

for one or more of the ethnic characteristics, taking at least some of the reference samples having a common ethnic characteristic together to give a grouping and considering the frequency of occurrence of the combination of the identity of the one or more variations at the one or more locations of the DNA for the sample with that ethnic characteristic.

The third aspect of the invention may further provide that the frequency of occurrence is used to predict information relating to the nature of the ethnic characteristic of the person who is the source of the sample.

The first and/or second and/or third aspects of the invention may further provide one or more of the following features, possibilities and options.

The ethnic characteristic may be an ethnic group. The ethnic groups may include one or more of White skinned European, Afro-Caribbean, Indo-Pakistani, South-East Asian, Middle Eastern. Other groups may be used separately from and/or together with such groups.

The source may be male or female. The source may be a suspect in a crime and/or a person linked to the scene of a crime and/or a person linked to an item implicated in a crime and/or linked to the scene of a crime.

The sample may be any DNA containing sample, such as a blood sample, a bodily fluid sample, skin sample, hair sample or the like. The sample may be taken from a location, such as a wall, floor, floor covering or the like, and/or from an item, such as furniture, an item of clothing or the like.

The sample may be analysed by DNA amplification based techniques. The analysis preferably analyses a plurality of locations simultaneously. Preferably the same type of analysis is undertaken for each location.

The method may consider at least 2, preferably at least 3, more preferably at least 4, still more preferably at least 6 locations, and ideally at least 10 locations.

The variation may be of the short tandem repeat type. The variation may thus include a number of different alleles which could occur at the location. The number of variations possible at a location may be 5, 10 or even more.

The locations may be a plurality of loci for the DNA, such as one or more selected from loci HUMVWFA31/A, HUMTH01, HUMFIBRA, D8S1179, D21 S11, D18S51, D3S1358, D2S1338, D16S539 or D19S433. Preferably the loci include at least three of HUMVWFA31/A, HUMTH01, HUMFIBRA, D8S1179, D21S11 or D18S51 and ideally at least four thereof. Additional information providing locations may be considered, such as sex indicating locations, for instance the X-Y homologous gene amelogenin.

The locations may be a plurality of loci for the DNA, such as one or more selected from loci HUMCD4, HUMPLA2A, HUMFIIDA, HUMAPOAI/1 OR HUMFABP. Preferably the loci include at least three of HUMCD4, HUMPLA2A, HUMFIIDA, HUMAPOAI/1 or HUMFABP, and ideally at least four thereof.

The loci may be any number of HUMVWFA31/A, HUMTH01, HUMFIBRA, D8S1179, D21S11, D18S51, D3S1358, D2S1338, D16S539 or D19S433 and ideally all 11; and/or include one, two, three or ideally all ten of D3S1358, D2S1338, D16S539 or D19S433; and/or include one, two, three, four or ideally all five of HUMCD4, HUMPLA2A, HUMFIIDA, HUMAPOAI/1 or HUMFABP. The variation may be of the single nucleotide polymorphism type.

The variation may be of the single nucleotide polymorphism (SNP) type. The variation may thus include a number of different bases which could occur at the location. The number of variations possible at a location may be two, three or four.

The locations may be at a plurality of loci for the DNA, such as one or more loci established as having SNPs which vary according to ethnic group to at least some extent.

The implication of the variation in ethnic characteristic prediction may be established by reviewing the variation with ethnic characteristics for a significant number of reference samples. For instance 200 or more samples from individuals having a given ethnic characteristic may be considered and the manner in which the variation occurrence and/or the identity of the variation changes with different ethnic characteristics occurs can be investigated. This may establish one or more locations and/or one or more variations at such locations as providing information relating to the ethnic characteristic of a sample source.

Preferably the database provides information on identity of the variations at the locations for which the sample is analysed, and ideally all of those locations. Preferably the nature of the physical characteristic is recorded with the information on variation. Preferably the database contains a number of reference samples which is statistically significant for the variations at the locations under consideration. The database may contain more than 200 or more than 500, or more than 1000, or preferably more than 5000 and ideally more than 10000 reference samples. Preferably the database contains at least 100, preferably at least 200, more preferably at least 500 and ideally more than 1000 reference samples for each potential nature of the physical characteristic, such as ethnic characteristic, under consideration and/or prediction.

Preferably the reference samples are randomly selected and/or are selected from a database of reference samples. Preferably the reference sample for each nature of the physical characteristic are randomly selected. The reference samples of the database as a whole and/or of one or more of the natures of the physical characteristic may be selected from a country population, a sub-set of a country population such as a regional population or location population or population based on other selection mechanisms such as other evidence.

Preferably the reference samples which are grouped together all have the same physical, such as ethnic, characteristic. The same physical characteristic may be the classification of the person in an ethnic group, such as White skinned European, Afro-Caribbean, Indo-Pakistani, South-East Asian or Middle Eastern. Preferably a reference sample in the database is grouped with all the other reference samples having a common nature therewith. Preferably the reference samples are only considered in one grouping of reference samples, ideally that grouping having a common nature.

Preferably the reference samples having a common physical characteristic, such as ethnic characteristic, are grouped and groups are formed for all the physical characteristics, such as ethnic characteristics, of the database. The frequency of occurrence of the identity of the one or more variations at one or more locations of the DNA of the sample in the grouping may thus be indicated for each of the physical/ethnic characteristics natures.

The frequency of occurrence of the combination of the presence and/or the identity of the variation at all of the locations may be provided. Preferably the frequency of occurrence of the combination of variations having that identity is considered. The frequency of occurrence of the variation having that identity may be considered against the frequency of occurrence of the combination of variations having that identity in the reference samples having a common nature for the physical characteristic. Preferably a plurality, ideally all, the variations are considered in this way against the reference samples, ideally all the reference samples, having a common nature for the physical characteristic considered. The frequency of occurrence of an allele at a variation may be considered in this way, ideally for all the variations.

The relative occurrence may be considered by a rules based calculation.

The calculation may be considered according to the formula:—

$$Likelihood = \frac{f in ethnic group A}{\begin{matrix}f in ethnic group B \times f in ethnic group C \times f \\ in ethnic group D \times f in ethnic group E\end{matrix}}$$

where f=the frequency of profile. The calculation may vary according to the number of ethnic groups under consideration. A likelihood value for each profile for each of the ethnic groups considered is preferably obtained. Preferably the likelihood values are compared to the number of likelihood distributions generated from samples of known ethnic origin.

The relative occurrence may be considered according to the formula:—

$$Posterior Prob . Pr ( A / G ) = \frac{Pr ( G / A )}{Pr ( G )} \times Pr ( A ) Prior Probability$$

where Pr (A/G) is the probability of the person from whom the sample sourced being of ethnic group (A) given that genotype (G) was revealed by the sample analysis; Pr (G/A) is the probability of genotype G occurring given the person is from ethnic group A; Pr (G) is the probability of genotype G from the whole suspect population, defined by Pr(G)=Pr(G/n₁).Pr(n₁)+Pr(G/n₂).Pr(n₂)+ . . . +Pr(G/nₓ).Pr(nₓ), where x is the number of different physical characteristic groups; Pr(A) prior probability is the proportion the ethnic group A represents of the whole suspect population A, B, C . . . x. In the formula the terms used may be changed as appropriate to calculate the probabilities for the other groups, other than (A) in an equivalent manner.

The frequency of occurrence of the combination for each of the groups may be considered to evaluate whether one ethnic group is more likely and/or less likely to be the source given the particular combination/genotype resulting from sample analysis.

The calculation according to the formula may be adjusted in the event of one of the identities of a variation being defined as a rare identity, for instance a rare allele, a rare identity being defined as those which occur within the sample under consideration, but which do not occur or occur only once in any one or all of the database groupings according to common nature of the physical characteristic. Preferably the calculation is only adjusted in relation to the location for which a rare identity is found. Preferably the adjustment involves the assigning of a fixed probability to the occurrence of that rare identity in the grouping from which it was missing and for which the frequency is less than 1/N*. Preferably the fixed probability is defined as 1/N*, with N* being the total number of alleles of at each locus, which is the same number for each locus, for which identity frequencies, for instance allele numbers, are available in the groupings of the database which has the lowest number of known samples which were used to generate that grouping in the database.

The information and/or prediction may be used to suggest that the person who is the source of the sample is a member of a particular ethnic group and/or is not a member of one or more ethnic groups or that an ethnic group cannot be predicted.

The information and/or prediction may be used to suggest a physical characteristic of a person as part of an elimination process, such as a criminal investigation. The information and/or prediction may be used to suggest the ethnic background of a person as part of an elimination process, such as a criminal investigation. The information and/or prediction may be provided to law enforcement or police authorities or the public to assist in the identification of persons, for instance suspects of a crime.

The information and/or prediction may be obtained by considering the frequency of occurrence in combination with other information of the potential source of the sample. The other information may be introduced to the relative occurrence consideration and/or may be considered together with the frequency of occurrence consideration to give overall information and/or an overall prediction.

According to a further aspect of the invention we provide a mixture for amplifying, preferably simultaneously, a plurality of loci, the loci including at least two of HUMCD4, HUMPLA2A, HUMFIIDA, HUMAPOAI/1, HUMFABP.

Preferably the mixture includes primers for all five of these loci. The mixture may include primers for one or more of loci HUMVWFA31/A, HUMTH01, HUMFIBRA, D8S1179, D21S11, D18S51, X-Y homologous gene amelogenin, D3S1358, D2S1338, D16S539 D19S433. Preferably the mixture is a multiplex.

Various embodiments of the invention will now be described, by way of example only.

In a variety of situations a DNA sample may be obtained without definitive evidence as to its source. The tracing of that source and/or the confirmation or rebuttal of an entity as being the source is a significant forensic tool.

A number of existing techniques consider a variety of features of the DNA of a sample and compare that with features in a sample from a known source to establish whether the sample arose form that source and the statistical confidence in reaching that conclusion. Such techniques do not provide much information about the source of the sample, however, before such a comparison is made.

In the technique of the present invention, however, analysis of a sample is used to determine a likely physical characteristic of the source of the sample. These characteristics can then be used to assist in identifying groups of the population as a whole for particular investigation and/or be used alongside other evidence to assist in the tracing of the source of the sample.

In one embodiment, the technique of the present invention involves the collection of a DNA sample from a crime scene in the conventional way for subsequent analysis. The analysis technique generates a DNA profile for the sample by considering the variations which occur at certain locations in the genes which make up the sample. The technique of considering a number of loci which exhibit short tandem repeat (STR) variation may be used for this purpose.

The applicant, for instance, regularly analyses DNA samples using six STR loci and a sex determinative locus. These loci are:—

i) HUMVWFA31/A;

ii) HUMTH01;

iii) HUMFIBRA;

iv) D8S1179;

v) D21S11;

vi) D18S51; and

vii) the X-Y homologous gene amelogenin.

This has recently been updated to add a further 4 STR loci, namely:—

viii) D3S1358;

ix) D16S539;

x) D2S1338;

xi) D19S433.

Where an unknown sample is under consideration, profiling using these STR loci would routinely be carried out for other investigative purposes, with the resultant profile also potentially being used in the technique of the present invention. If other STR loci are to be investigated, then those may be specifically investigated for the technique of the present invention.

To date the profile generated has been compared with individual samples in a database, for instance the DNA profile database operated by The Forensic Science Service in the UK, The National DNA Database (Registered Trade Mark). Highly similar matches between the unknown sample and a sample in the database can then be used to indicate that the source of that sample should be considered further as the particular source of the unknown origin sample.

The present invention, however, uses the DNA profile generated for the unknown source sample in a different way. The DNA profile of the sample provides an indication as to which particular allele the DNA of the sample possesses at each of the loci under investigation. Some of these alleles may be relatively common to the population, whereas some may be relatively unusual.

In addition to the analysis of the sample of unknown origin the technique also requires a database containing a significant number of DNA profiles from at least partially known origins. The compilation of this database involves the analysis of the DNA from the known source to determine its allele variation at the loci under consideration. The variation in alleles which occurs is recorded together with the ethnic group of the person providing the sample. In general the ethnic groupings used are white skinned Europeans, Afro-Caribbeans, Indo-Pakistanis, South-East Asians and Middle Easterners.

Once collected the results for the various ethnic groups can be considered to determine the frequency of occurrence of the various alleles variations at the loci considered for that ethnic group as a whole, subject to the incorporation of size bias and corrections. Significant variation between the groups occurs with, for instance a particular allele variation being common in one group, but relatively rare in one or more of the others. For instance, such variations for the STR locus HUMFIBRA and allele 18.2 are listed in Table 1.

TABLE 1

| | | | | South | |
|---|---|---|---|---|---|
| | White | Afro- | Indo- | East | Middle |
| | Caucasian | Caribbean | Pakistani | Asian | Eastern |
| Numbers | 18.2 | 5 | 145 | 0 | 0 |
| Total HUMFIBRA | 119882 | 11380 | 4162 | 1234 | 1934 |
| alleles | | | | | |
| Frequency of 18.2 | 0.0002514 | 0.0127416 | 0 | 0 | 0 |

The frequency in this Table does not include the size bias correction.

The relative frequency of the ethnic groups to one another is also included when making the analysis.

As an example of the applicability of this technique reference is made to the following pilot study.

For a single police region in the UK, 176 DNA profiles which had been collected by the police force in the usual way and had been submitted for matching with individual records in the database operated by The Forensic Science Service were considered. Whilst the ethnic grouping of each of these samples was known to the police force in question, the processing and analysis of the samples was conducted blind prior to comparison of the predicted ethnic groups with the actual ethnic groups.

As stated above the samples were analysed using an STR based technique to obtain a DNA profile in each case. The alleles occurring were compared with the frequency of occurrence information for the various alleles for the various loci with each of the different ethnic groups using a “rules” based calculation.

For a DNA profile of unknown ethnic origin, the frequency of the profile in each of the five ethnic groups was calculated as according to the technique described in more detail below. In order to determine the most likely ethnic group for the profile's origin, a likelihood value was generated as follows:

Likelihood=frequency of profile (f) in ethnic group A divided by f in group B times f in group C times f in group D times f in group E.

This calculation yields five likelihood values for each profile, namely the likelihood of the profile being from a person in ethnic group A or ethnic group B or ethnic group C or ethnic group D or ethnic group E. These values are then compared to a database of previously calculated values that have been obtained from samples of known ethnic origin. These known ethnic origin samples are used to produce a distribution and the likelihood value from the calculation is compared to the 95^{th}, 100^{th} and 10 times 100^{th} upper and lower percentile ranges of the 25 distributions.

The relative location of the unknown profiles calculated likelihood values within the distributions determine the most likely ethnic origin of that sample.

The results of the statistical comparison was used to give one or more of a number of different predictions depending upon the nature of the result. These prediction types included:—

in) those cases where a major ethnic group, a major ethnic group being either white skin European, afro-Caribbean or indo-Pakistani, was indicated as being statistically the source compared with the other groups;

ii) those cases where an major ethnic group a major ethnic group being either white skin European, afro-Caribbean or indo-Pakistani, could be excluded as statistically being the source compared with the other groups;

iii) those cases where no ethnic group could be suggested as more applicable than the others.

For the 176 samples the following predictions were made.

| | Percentage of predictions being this |
|---|---|
| Prediction Type | type |
| major ethnic group indicated | 27% |
| major ethnic group excluded | 35% |
| no ethnic group assignable | 38% |

For the 176 samples, therefore, a useful prediction which could be used to help trace the source was obtained in 109 cases. When the predictions were compared with the known information of the sources only 7 of the 109 predictions were found to be incorrect. Subsequently 3 of those 7 were established as arising from DNA samples from an item with which the alleged known person was unrelated and were thus void considerations. Only 4 out of the predictions were thus incorrect, an error of 2.3% of the total cases considered. As the technique is statistically based some errors are likely to occur.

As an alternative to the “rules” type calculation conducted above it is possible to use alternative formula for the calculations. This consideration is based around formula I given below, in this case expressed as, Pr (A/G), the probability of the person from whom the sample sourced being of ethnic group (A) given that genotype (G) was revealed by the sample analysis and three ethnic groups (A,B,C) are under consideration, where

a) Pr (G/A) is the probability of genotype G occurring given the person is from ethnic group A;

b) Pr (G) is the probability of genotype G from the whole suspect population, defined by Pr(G)=Pr(G/A).Pr(A)+Pr(G/B).Pr(B)+Pr(G/C).Pr(C);

c) Pr (A) prior probability is the proportion the ethnic group A represents of the whole suspect population A, B and C.

$$Posterior Prob . Pr ( A / G ) = \frac{Pr ( G / A )}{Pr ( G )} \times Pr ( A ) Prior Probability \qquad Formula I$$

Similar calculations can be calculated for the sample source being of ethnic group (B) given genotype (G) and the sample source being of ethnic group (C) given genotype (G). The three relative probabilities can then be considered to evaluate whether one ethnic group is far more likely and/or far less likely to be the source given the genotype (G).

In the above presentation of the formula I, the value of Pr(G/A) is in effect the product of the relative proportion of each of the possible alleles which occurs at each loci in ethnic group A. As the loci may provide heterozygous variation (for example locus THO1 where the alleles 9 and 9.3 may be found, the allele being inherited from each parent being different) or homozygous variation (for example locus THO1, for allele 7 where the alleles inherited from each parent are the same two modes of calculation are employed. For individual allele proportions at heterozygous loci, p or q=(occurrence in database+1)/(database size+2). For individual alleles at homozygous loci, p=(occurrence in database+2)/(database size+2). The overall genotype probability is thus calculated by multiplying all the allele proportions together (factored by 2 for heterozygous alleles, i.e. for heterozygous locus frequency of alleles at that locus=²p.q, for homozygous locus frequency of alleles at that locus=p²).

Whilst this basic form can be used in the application of formula I, a more balanced consideration is achieved where the impact of the occurrence of rare alleles in the analysed sample is taken into account.

Rare alleles are taken as those which occur within the profile under consideration, but which do not occur or occur only once in any one or all of the ethnic grouping databases. Thus if allele H has not been found before in any of the known samples which make up the ethnic database for ethnic grouping A then that allele H is considered a rare allele.

Rare allele compensation is preferably only applied to the locus for which a rare allele is identified and aims to provide an alternative allele frequency calculation so as to avoid a database size bias problem. Due to certain ethnic groups being smaller proportions of the population, and particularly due to the smaller size of the comparison databases used for these ethnic groups, the correction is needed to avoid the above mentioned Pr(G/A) type calculation biassing the prediction towards the smaller ethnic group or groups.

The rare allele compensation method provides that a minimum proportion value of 1/N* be applied for that rare allele in each of the ethnic group frequency of occurrence sets, with N* being the total number of alleles at that locus for which allele frequencies are available in the ethnic group database which has the lowest number of known alleles which were used to generate that database. Thus N*=550 where allele H does not occur in the frequency of occurrence database for ethnic group A, when the frequency of occurrence databases were generated using 1500, 350 and 275 known samples for ethnic groups A, B and C respectively and hence ethnic group C has 550 alleles detected in the 275 known samples for all loci.

Formula I, particularly in its precise forms, is flexible in that it allows the relative levels of persons in the various ethnic groups to be taken into account when making the prediction. Whilst these could be the relative levels of those ethnic groups in the world population or country population, they could equally reflect a suspect population and/or take into account other evidence sources such as eyewitness accounts.

Whilst the invention is described above in relation to STR based techniques for six loci, other loci could be used to supplement this investigation and/or to investigate completely different loci.

Four additional loci particularly suitable for investigation purposes are

1) D3S1358;

2) D2S1338;

3) D16S539;

4) D19S433.

Five additional or further additional loci particularly suitable for investigation purposes, as they relate to loci which have alleles which are particularly variable between two or more of the ethnic groups are:—

1) HUMCD4;

2) HUMPLA2A;

3) HUMFIIDA;

4) HUMAPOAI/1;

5) HUMFABP.

Furthermore, whilst the technique has been described in relation to comparison of STR analysis of an unknown source sample with frequency of occurrence information for allelic variation at those loci for different ethnic groups, other variations could be considered, such as SNP's, where the frequency of variation or of a particular variation at a site with different ethnic groups varies. The use of such an alternative variation would involve considering a number of samples whose ethnic group or other characteristic was known to determine what variations and/or what identity occurs at what variations for those samples. As a consequence, different likelihood of occurrence of a variation and/or an identity of a variation could be established for different ethnic groups or other characteristics. The variation and/or identity of variation of an unknown sample can then be compared to establish a prediction for its ethnic group or other characteristic based on how that unknown sample's variations and/or identities of variations correspond to the probabilities for the variations and/or identities of variations established for the reference samples.

1. A method of obtaining information about the nature of a physical characteristic of the source of a sample from a number of possibilities for that physical characteristic, the method comprising
analysing at least part of the DNA in the sample, the analysis determining the presence and/or identity of one or more variations at one or more locations of the DNA;
providing a database containing information on the presence and/or identity of the one or more variations at the one or more locations of the DNA for a plurality of reference samples, the nature of the physical characteristic being known for the reference samples;
for one or more of the possible natures of the physical characteristic, taking at least some of the reference samples having a common nature for the physical characteristic together to give a grouping and considering the frequency of occurrence of the combination of the presence and/or identity of the one or more variations at the one or more locations of the DNA for the sample in that grouping having a common nature of the physical characteristic;
the frequency of occurrence being used to predict information relating to the nature of the physical characteristic of the source of the sample.

2. A method according to claim 1 in which the physical characteristic is the ethnic characteristic of the sample's source.

3. A method according to claim 1 in which the frequency of occurrence of those variations with ethnic characteristics is considered.

4. A method of obtaining information about the ethnic characteristic of a person who is the source of a sample, from a number of possible ethnic characteristics, the method comprising
analysing at least part of the DNA in the sample, the analysis determining the identity of one or more variations at one or more locations of the DNA;
providing a database containing information on the identity of the one or more variations at the one or more locations of the DNA for a plurality of reference samples taken from people whose ethnic characteristic is known and recorded in the database;
for one or more of the ethnic characteristics, taking at least some of the reference samples having a common ethnic characteristic together to give a grouping and considering the frequency of occurrence of the combination of the identity of the one or more variations at the one or more locations of the DNA for the sample with that ethnic characteristic;
the frequency of occurrence being used to predict information relating to the nature of the ethnic characteristic of the person who is the source of the sample.

5. A method of obtaining information about the nature of a physical characteristic of the source of a sample from a number of possibilities for that physical characteristic, the method comprising
analysing at least part of the DNA in the sample, the analysis determining the presence and/or identity of one or more variations at one or more locations of the DNA;
providing a database containing information on the presence and/or identity of the one or more variations at the one or more locations of the DNA for a plurality of reference samples, the nature of the physical characteristic being known for the reference samples;
for one or more of the possible natures of the physical characteristic, taking at least some of the reference samples having a common nature for the physical characteristic together to give a grouping and considering the frequency of occurrence of the combination of the presence and/or identity of the one or more variations at the one or more locations of the DNA for the sample in that grouping having a common nature of the physical characteristic to obtain the information about the nature of the physical characteristic of the source of the sample;
the frequency of occurrence being used to predict information relating to the nature of the physical characteristic of the source of the sample.

6. A method according to claim 1 in which the ethnic characteristic is an ethnic group, the ethnic groups including one or more of White skinned European, Afro-Caribbean, Indo-Pakistani, South-East Asian, Middle Eastern.

7. A method according to claim 1 in which the locations are a plurality of loci for the DNA, including one or more selected from loci HUMVWFA31/A, HUMTH01, HUMFIBRA, D8S1179, D21S11, D18S51, D3S1358, D2S1338, D16S539 or D19S433.

8. A method according to claim 1 in which the database contains more than 200 reference samples for the variations at the locations under consideration.

9. A method according to claim 1 in which the database contains at least 100 reference samples for each potential nature of the physical characteristic, such as ethnic characteristic, under consideration and/or prediction.

10. A method according to claim 1 in which the reference samples having a common physical characteristic, such as ethnic characteristic, are grouped and groups are formed for all the physical characteristics, such as ethnic characteristics, of the database, the frequency of occurrence of the identity of the one or more variations at one or more locations of the DNA of the sample in the grouping being indicated for each of the physical/ethnic characteristics natures.

11. A method according to claim 1 in which the frequency of occurrence of the variation having that identity is considered against the frequency of occurrence of the combination of variations having that identity in the reference samples having a common nature for the physical characteristic.

12. A method according to claim 1 in which the likelihood of occurrence of that combination of variables with a physical characteristic is calculated according to the formula:—
$$Likelihood = \frac{f in ethnic group A}{\begin{matrix}f in ethnic group B \times f in ethnic group C \times f \\ in ethnic group D \times f in ethnic group E\end{matrix}}$$
where f=the frequency of profile A, B, C, D and E are particular ethnic groups, A being the ethnic group corresponding to that physical characteristic.

13. A method according to claim 1 in which a likelihood value for each profile for each of the ethnic groups considered is obtained.

14. A method according to claim 1 in which the frequency of occurrence of the combination for each of the groups may be considered to evaluate whether one ethnic group is more likely and/or less likely to be the source given the particular combination/genotype resulting from sample analysis.

15. A method according to claim 1 in which the calculation is adjusted in the event of one of the identities of a variation being defined as a rare identity, for instance a rare allele, a rare identity being defined as those which occur within the sample under consideration, but which do not occur or occur only once in any one or all of the database groupings according to common nature of the physical characteristic.

16. A method according to claim 1 in which the adjustment involves the assigning of a fixed probability to the occurrence of that rare identity in the grouping from which it was missing and for which the frequency is less than 1/N*, with N* being the total number of alleles at each locus, which is the same number for each locus, for which identity frequencies, for instance allele numbers, are available in the groupings of the database which has the lowest number of known samples which were used to generate that grouping in the database.

17. A method according to claim 1 in which the information and/or prediction is used to suggest that the person who is the source of the sample is a member of a particular ethnic group and/or is not a member of one or more ethnic groups or that an ethnic group cannot be predicted.
